(19) United States
(12) Patent Application Publication
(10) Pub. No.: US 2019/0125479 A1
(43) Pub. Date: May 2, 2019

(54) FIT-CHECK DEVICE

(71) Applicant: Q30 SPORTS SCIENCE, LLC, Westport, CT (US)

(72) Inventors: Jamison Float, Westport, CT (US); James Lua, Westport, CT (US); Dan Wisniewski, Westport, CT (US); Brandon Eder, Westport, CT (US); Gary Myers, Westport, CT (US); Sanro Zlobec, Westport, CT (US)

(21) Appl. No.: 15/974,996

(22) Filed: May 9, 2018

Related U.S. Application Data
(63) Continuation of application No. PCT/US16/62006, filed on Nov. 15, 2016.
(60) Provisional application No. 62/256,004, filed on Nov. 16, 2015.

(51) Int. Cl. A61B 90/00; A61B 17/12; G01L 5/00

(52) U.S. Cl. CPC A61B 90/06; A61B 2017/00115; G01L 5/0028; A61B 17/12

(57) ABSTRACT

The disclosure provides a fit-check device and associated methods. In some embodiments, the device may be used in conjunction with a rigid or semi-rigid partially circumferential concussion-mitigating collar to determine whether the collar is appropriately sized for the user and is applying the desired amount of force to the neck veins.

### CROSS-REFERENCE TO RELATED APPLICATIONS

The subject application is a continuation of PCT Patent Application No. PCT/US16/62006, filed Nov. 15, 2016, which claims priority to U.S. Provisional Patent Application No. 62/256,004, filed on Nov. 16, 2015, both of which are incorporated by reference herein in their entirety for all purposes.

### FIELD OF THE INVENTION

The present application discloses a fit-check device and associated methods. In some embodiments, the device may be used in conjunction with a rigid or semi-rigid partially circumferential concussion-mitigating collar to determine whether the collar is appropriately sized for the user and is applying the desired amount of force to the neck veins.

### BACKGROUND

Traumatic brain injury (TBI) continues to be one of the most common causes of death and morbidity in persons under age 45, even in western societies. A reported 1.7 million people suffer from TBI annually in the United States alone, resulting in an estimated per annum total cost of over $60 billion. Historically, prevention of skull and brain injury has focused on the use of helmets as external cranial protection. Although headgear is effective in preventing the most devastating intracranial injuries—penetrating injuries and skull fractures—headgear is somewhat limited in its ability to reduce instances of concussions or damage to the structures within the cranium. The vast majority of concussions and other traumatic brain injuries occur when a person is subjected to high velocity acceleration-deceleration mechanisms which are not sufficiently mitigated by helmets. Such forces also are known to cause injury to the internal structures of the ear, eye, and nose. These injurious forces may be imparted when the body suffers a violent collision, such as in a car accident or during a contact sport such as football, or when the body is subjected to a blast wave. In large part, relative freedom of movement of the human brain within the cranial cavity predisposes it to both linear and rotational force vectors, with resultant energy absorption causing cellular disruption and dysfunction, sometimes with delayed cell death.

It has been discovered that injuries to the brain, eye, ear, and nose caused by concussive forces and blast waves may be reduced or prevented by moderately increasing the intracranial blood pressure. Fully-circumferential and partially circumferential collars designed to be worn about the neck and that apply pressure to the internal jugular vein(s), external jugular vein(s), and/or other neck veins have been designed. These collars are typically designed to apply about 5-80 mm Hg pressure to the neck veins.

In some embodiments, the disclosed invention provides a device and associated methods that determines whether a partially circumferential collar is appropriately sized for the subject and/or that it applies the appropriate amount of pressure to the targeted neck veins in order to reduce or prevent the incidence of a concussion or other traumatic (but non-penetrating) brain injury.

### SUMMARY OF THE INVENTION

The invention provide devices, systems, and associated methods for testing the fit and/or function of a partially circumferential concussion-mitigating collar.

In one aspect, the invention provides a fit-check device having a force-check feature. In one embodiment, the fit-check device includes a housing having a first rigid side and an opposing second rigid side, a pressure sensor positioned on the first rigid side, a pressure indicator, and a pressure transducer configured to receive a pressure input signal from the pressure sensor and transmit a pressure output signal to the pressure indicator. Optionally, the fit-check device also contains a battery, an electrical circuit, a microprocessor, a printed circuit board assembly, a memory unit, and/or a radio transceiver. In some embodiments, the pressure indicator includes one or more light emitting diodes (LEDs), an audio speaker, and/or a vibration generating device.

In operation, one terminal of the partially circumferential collar is contacted to one rigid side, and the second terminal of the collar is contacted to the pressure sensor. The pressure sensor detects the amount of inwardly-directed force applied by the collar. The pressure transducer receives the pressure input signal from the pressure sensor, compares that signal to one or more predetermined values, and then transmits a pressure output signal to the pressure indicator, which displays a signal or other information based on the amount of force applied to the pressure sensor. Optionally, the opposing second rigid side contains a second pressure sensor, and the pressure input signal from both pressure sensors is integrated at the pressure transducer to indicate the total amount of inwardly-directed pressure exerted by the collar. The distance between the first and second opposing sides is adapted to gauge/measure the amount of inwardly-directed pressure exerted by the collar terminals on the subject's neck when the collar device is worn and is appropriately-sized for the subject's neck. For example, the distance between the first and second opposing sides is approximately equal to the designed gap between the collar terminals when the collar is worn. Optionally, the fit-check device also has a collar size selector function in which the operator can select on the fit-check device the collar size being tested. The collar size selector function integrates the selected collar size with the pressure measured by the pressure sensor(s) to compute and signal an output. For example, collars with smaller design gaps between the collar terminals may be expected to exert a high pressure on the fit-check device than collars with larger design gaps even though both collars may apply approximately the same amount of pressure on the subject's neck, when worn and properly sized for the subject.

In one embodiment, the pressure indicator illuminates an LED, sounds an audible tone, and/or vibrates when the force applied to the pressure sensor is greater than a predetermined amount of force. In a related embodiment, the pressure output signal is transmitted to a second device (e.g., a smart phone, tablet, laptop, or desktop computer), and that second device displays the signal or other information based on the amount of force applied to the pressure sensor. The output display on the second device may be included instead of, or in addition to, the display provided by the pressure indicator.

In another aspect, the invention provides a fit-check device having a size-check feature. The size-check feature may be incorporated into a fit-check device that contains or does not contain a force-check feature. In one embodiment, the size-check feature includes a measuring device on one side or face of a fit-check device. The measuring device may be a ruler or a color bar. Color bars optionally have five colored segments. In one configuration, the first, third, and fifth segments are commonly colored in a first color (e.g., red), and the second and fourth segments are commonly colored in a second color (e.g., green). The distance between the second and fourth segments (i.e., the length of the third segment) is based on the length of the gap between collar terminals when the collar is worn by, and is properly fit to, the subject. For example, the distance between the centers of the second and fourth segments is equal to the gap. The width of those segments represents the tolerance or inter-individual variability expected or allowed while still maintaining a proper and functional collar fit.

In another embodiment, the size-check feature includes (i) two sensors positioned on a side of the fit-check device, wherein the sensors are separated by a predetermined space, and (ii) a fit indicator configured to provide a user with an indication of whether the two sensors are simultaneously activated. In some embodiments, the fit indicator includes one or more light emitting diodes (LEDs), an audio speaker, and/or a vibration generating device. In operation, the collar is donned by the subject, and the collar terminals are individually aligned with the sensors. The sensors are operably connected to the fit indicator, which provides a signal when both sensors are simultaneously contacted or activated by the individual collar terminals. In a related embodiment, the sensors' output signal is transmitted to a second device (e.g., a smart phone, tablet, laptop, or desktop computer), and that second device displays the signal. The output display on the second device may be included instead of, or in addition to, the display provided by the fit indicator.

In some embodiments of any of the fit-check devices described herein, the fit-check devices optionally include a data communication device and/or a memory unit. This configuration is particularly useful to retrieve and store data obtained from sensors built into the collars. Optionally, the memory unit also stores fit-check information and associates that information with the sensor data for a particular collar. Suitable data communication devices include, but are not limited to, data ports (e.g., USB ports) and wireless receivers, transmitters, and transceivers. The memory unit (e.g., RAM, flash memory, etc.) may store digital and electronic information in any convenient manner.

In some embodiments, the size-check feature is performed by a touch-screen device executing a software application as described herein. In other embodiments, the size-check feature is executed by a device having a camera and executing a software application as described herein.

By “pressure sensor” is meant a pressure sensor known in the art as well as any suitable type of force sensor, contact switch, calibrated spring, or equivalent device.

By “subject” or “user” is meant the individual who is wearing the partially circumferential collar.

By “operator” is meant the individual who is interpreting the output of the fit-check device. The operator may be the subject or another party.

Accordingly, a non-limiting aspect of the present invention seeks to provide a device, comprising:
an input device for capturing a distance between first and second terminals of a TBI-mitigating collar;
a processor configured for determining whether the distance meets predefined size criteria for the TBI-mitigating collar and for outputting via an output device a signal when the distance is determined to meet the predetermined size criteria for the TBI-mitigating collar.

Another non-limiting aspect of the present invention seeks to provide a device, comprising:
a first contact region for receiving a first terminal of a TBI-mitigating collar;
a second contact region for receiving a second terminal of the TBI-mitigating collar;
a pressure sensor connected to at least one of the first and second region, for determining a pressure applied by the TBI-mitigating collar;
a processor configured for determining whether the applied pressure falls in a predetermined range for the TBI-mitigating collar and for outputting via an output device a signal dependent on the determining.

Another non-limiting aspect of the present invention seeks to provide a system, comprising:
a TBI-mitigating collar; and
a device for determining a fit of the TBI-mitigating collar.

Another non-limiting aspect of the present invention seeks to provide a device for determining a fit of a TBI-mitigating collar, comprising:
a pressure sensor for determining a pressure applied by the TBI-mitigating collar while the latter is connected to the device;
size measurement means for determining a dimension of the TBI-mitigating collar while the latter is worn by a subject;
a processor for outputting an indication of fit based on at least one of the applied pressure or determined dimension.

Another non-limiting aspect of the present invention seeks to provide a device, comprising:
an input device for receiving an indication of a particular TBI-mitigating collar and for capturing a distance between first and second terminals of the particular TBI-mitigating collar;
a memory storing size criteria for a plurality of TBI-mitigating collars including said particular TBI-mitigating collar;
a processor configured for determining whether the distance meets the size criteria for the particular TBI-mitigating collar and for outputting via an output device a signal when the distance is determined to meet the size criteria for the particular TBI-mitigating collar.

Another non-limiting aspect of the present invention seeks to provide a device, comprising:
an input device for receiving an indication of a particular subject and for obtaining at least one of (i) a distance between first and second terminals of a TBI-mitigating collar while worn by the particular subject and (ii) a pressure applied by the TBI-mitigating collar to the device;
a memory storing TBI-mitigating device criteria for a plurality of subjects including said particular subject;
a processor configured for determining whether the distance and/or the pressure meets the TBI-mitigating device criteria stored in the memory and for outputting via an output device a signal that depends on the determining.

Another non-limiting aspect of the present invention seeks to provide non-transitory computer-readable storage media storing computer-readable instructions which, when executed by a processor of a device, cause the device to carry out a method that includes:
being responsive to an indication of a measured gap size between terminals of a TBI-mitigating collar;
obtaining from the storage media a predetermined gap size for the TBI-mitigating collar;
comparing the measured gap size to the appropriate gap size;
causing the device to output a perceptible signal based on an outcome of the comparing.

Another non-limiting aspect of the present invention seeks to provide non-transitory computer-readable storage media storing computer-readable instructions which, when executed by a processor of a device, cause the device to carry out a method that includes:
being responsive to an indication of a measured pressure applied by terminals of a TBI-mitigating collar at a certain gap distance;
obtaining from the storage media a predetermined pressure or pressure range for the TBI-mitigating collar;
causing the device to output a perceptible signal based on an outcome of utilizing the measured pressure and the predetermined pressure or pressure range.

Another non-limiting aspect of the present invention seeks to provide a method of checking a fit of a TBI-mitigating collar, comprising:
measuring a gap distance between terminals of the TBI-mitigating collar while the latter is worn by a subject;
measuring a pressure applied by the terminals of the TBI-mitigating collar at a predetermined gap distance between the terminals;
comparing the gap distance and pressure measurements to predetermined fit criteria to assess suitability of the TBI-mitigating for the subject.

Another non-limiting aspect of the present invention seeks to provide a device, comprising:
an input device for capturing a distance between first and second terminals of a TBI-mitigating collar;
a processor configured for determining whether the distance meets predefined size criteria for the TBI-mitigating collar and, if not, for outputting via an output device a suggestion for an adjustment to the TBI-mitigating collar.

Another non-limiting aspect of the present invention seeks to provide a device, comprising:
an input for receiving, from a TBI-mitigating collar fitted to a subject, an indication of pressure of the collar being applied to a neck of a subject
a processor configured for comparing the applied pressure to at least one predetermined pressure value to as to determine correctness of the fit of the TBI-mitigating collar.

Another non-limiting aspect of the present invention seeks to provide a method, comprising:
using a networked wireless communication device to determine at least one of a size measurement and a pressure measurement of a TBI-mitigating collar.

### BRIEF DESCRIPTION OF THE DRAWINGS

FIG. 1 illustrates a partially circumferential collar that may be used in conjunction with a fit-check device described herein, and the proper placement of that collar on the neck of a subject.

FIG. 2 illustrates the layout of one embodiment of a segmented measuring device.

FIG. 3A illustrates the use of a fit-check device for checking that the size of the partially circumferential collar is appropriate for the neck size of the subject. As illustrated, the collar size is appropriate.

FIG. 3B illustrates the use of a fit-check device for a collar that is too large for the neck size of the subject.

FIG. 3C illustrates the use of a fit-check device for a collar that is too small for the neck size of the subject.

FIG. 3D illustrates another use of a fit-check device to determine whether the collar size is appropriate for the subject.

FIG. 4A illustrates the interaction between a partially circumferential collar and a fit-check device when determining the neck vein pressure expected to be applied by the collar when worn.

FIG. 4B illustrates another use of a fit-check device to determine whether the collar applies a sufficient amount of neck vein pressure.

FIG. 5 provides an exploded view of a fit-check device.

FIG. 6 provides another exploded view of a fit-check device.

FIG. 7 is a block diagram illustrating certain operational components of a fit-check device, in accordance with a non-limiting embodiment.

FIG. 8 is a flowchart illustrating operation of a size-check process executed by a fit-check device, in accordance with a non-limiting embodiment.

FIG. 9A is a block diagram illustrating certain operational components of a fit-check device, in accordance with another non-limiting embodiment.

FIG. 9B illustrates transmission of messages between the fit-check device of FIG. 9A and a server.

FIG. 10 illustrates a collar including a marking having a known size.

FIG. 11 is a flowchart illustrating operation of a size-check process executed by a fit-check device, in accordance with another non-limiting embodiment.

FIG. 12 shows a database including a plurality of records that associate gap sizes with corresponding subject identifiers, in accordance with a non-limiting embodiment.

FIG. 13 illustrates a distance between terminals of a collar donned by a subject and a distance between jugular veins of the subject.

FIG. 14 is a flowchart illustrating operation of a force-check process executed by a fit-check device, in accordance with a non-limiting embodiment.

FIG. 15 shows a smartphone being used to detect pressure applied by extremities of a TBI-mitigating collar, in accordance with a non-limiting embodiment.

FIG. 16A shows a smartphone being used in conjunction with a spacer to detect pressure applied by extremities of a TBI-mitigating collar, in accordance with a non-limiting embodiment.

FIG. 16B shows back-to-back smartphones being used to detect pressure applied by extremities of a TBI-mitigating collar, in accordance with a non-limiting embodiment.

FIG. 17 is a block diagram showing communication between a TBI-mitigating collar, a fit-check device and a central server, in accordance with a non-limiting embodiment.

FIG. 18 shows a screen with two buttons representing an appropriate gap size, in accordance with a non-limiting embodiment.

### DETAILED DESCRIPTION

Various embodiments or examples may be implemented in numerous ways, including as a system, a process, or an apparatus/device. The invention is described in terms of various features and functionalities. It is understood that any particularly claimed invention may incorporate any one or more of the disclosed features and functionalities in any order or combination. The detailed description and figures are provided to facilitate an understanding of the invention, but are not intended to be limiting in any way.

### Partially Circumferential Collars

Any rigid or semi-rigid partially circumferential collar designed to be worn about the neck of a subject may be used in conjunction with the devices and methods described herein. In particular, partially circumferential collars designed to apply about 5-80 mm Hg pressure to the neck veins have been described in U.S. Pat. Nos. 9,168,045, 9,173,660, U.S. Patent Publication Nos. 2014/0142616 and 2014/0343599, and PCT Publication Nos. WO 2012/054262 and WO 2013/055409, each of which is hereby incorporated by reference in its entirety.

Partially circumferential collars are designed to encircle a portion of the neck of the wearer (e.g., preferably more than half of the neck circumference) and apply pressure to one or more neck veins (e.g., the internal jugular vein(s) and/or the external jugular vein(s)). Optionally, pressure is applied to the neck veins via inflatable or non-inflatable protuberances that are positioned on the collar such that they contact the skin immediately overlying the target vein(s). Neck-vein pressure is applied by an inwardly directed force exerted by the collar itself either directly on the skin or via the protuberances. Optionally, the protuberances are adapted to exert an independent or additional force including, for example, force applied by inflatable protuberances.

A partially circumferential collar is defined by an opening, gap, or discontinuity in the substantially circular shape of the collar. Although the collar may be designed such that the opening may fall anywhere on the circumference of the neck, openings that span the laryngeal prominence are preferred because they improve comfort and donning of the collar. As discussed in more detail below, an opening at the front of the neck is preferred but is not essential to the use of the fit-check device. Openings at the side and back of the collar are also compatible with the use of the fit-check device, particularly when audible or vibrational signals are used. In one embodiment, the collar has a core of memory metal or other spring-like material which allows the collar to be partially deformed during donning and doffing processes while retaining its shape and compressive properties.

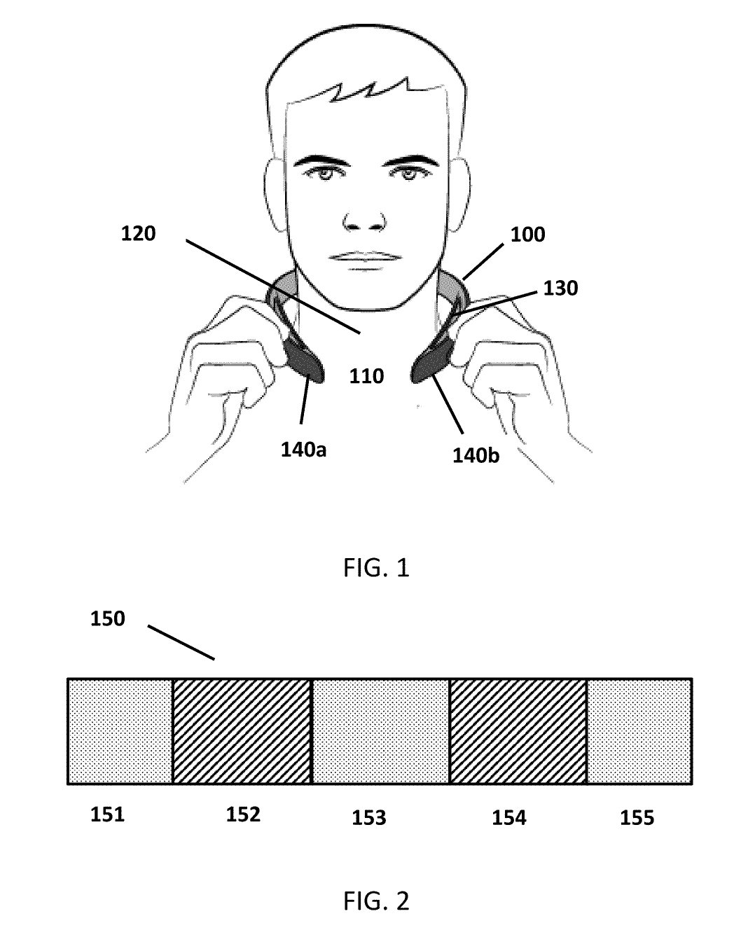

FIG. 1 illustrates a partially circumferential collar 100 that is suitable for use in connection with the fit-check device. The collar 100 is illustrated as having a gap 110 that spans the laryngeal prominence 120 of the subject. Protuberances 130 are positioned on the collar to contact the skin overlying the jugular veins and apply pressure thereto. The ends of the collar 100 are defined by the collar terminals 140 which may be contiguous with the protuberances and/or apply force directly to the neck veins.

TBI mitigation is a biological effect resulting from an elevated intracranial pressure. Thus, it is essential that the collar 100 is appropriately sized to fit the subject's neck and has sufficient resiliency and compressive force to apply the desired amount of pressure on the neck veins. Properly functioning but ill-fitting collars may not provide the desired protection. For example, collars having too large a circumference for the user's neck will not stretch the resilient collar material enough to achieve the desired pressure. Alternatively, collars having too small a circumference may apply too much pressure to the neck veins, or the pressure-applying collar surfaces will not properly align with the neck veins. Furthermore, many resilient materials suitable for use in the collar (e.g., memory metal) have deformation limits beyond which the material loses it resilience or “memory” and, therefore, its ability to apply an inwardly-directed force to the neck veins. The collar's resiliency may be lost through normal wear (i.e., cyclic use associated with donning and doffing) and as a result of normal material fatigue. Resiliency also may be lost if the collar is “over-stretched” or opened up beyond its design limits as may occur if the user dons the collar over its head rather than sliding it around the neck. Alternatively, over-stretching may occur if a donned collar is pulled away from the subject's neck as might occur during a contact sport like football. Often, the collar will not be perceptibly deformed even though it has lost its resiliency. Accordingly, there is a need to provide a device that is capable of objectively assessing a collar's fit and/or ability to apply an inwardly-directed compressive force.

In some embodiments, the collars are fitted with one or more sensors and a memory unit to record and store the sensor data. Sensors that may be incorporated into the collar include, for example, sensors that measure acceleration (e.g., an accelerometer), blood pressure, heart rate, galvanic skin response, neural activity, and/or skin temperature. Incorporation of the sensors into the collar may be done according to the principles and techniques commonly used in the construction of wearable fitness trackers such as those that are commercially available from Jawbone/Aliphcom (including Body Media) and FitBit. Optionally, the collars further contain a data communication device so that the sensor data may be exported to another computing device. Suitable data communication devices include, for example, a data port (e.g., a USB port) and a wireless transmitter or transceiver (e.g., Bluetooth, WiFi, cellular, or NFC). Optionally, the collar may contain a unique identifier that can be read or detected by the fit-check device in order for the device to associate sensor data and/or fit-check data with a particular collar in the memory unit of the fit-check device. Suitable identifiers include, for example, a bar code (for embodiments in which the fit-check device has a barcode reader), an RFID tag, and the like.

### Fit-Check Device Construction and Functionality

In some embodiments, the invention provides a fit-check device 300 that is capable of measuring the gap 110 in a partially circumferential collar to ensure proper sizing (“size-check”), the compressive force applied by the collar (“force-check”), or both. Optionally, the fit-check device 300 is capable of retrieving, storing, and transmitting sensor data from one or more collars fitted with sensors, and uniquely identifying each sensor data set with each collar or subject. Optionally, the fit-check device 300 is capable of storing and transmitting fit-check information (i.e., information relating to the size-check and force-check features) for one or more collars. Preferably, the fit-check information is associated with the sensor data in the memory unit and suitable for transmission. In one embodiment, the subject retrieves sensor data from the collar and stores that data in the memory unit of the fit-check device. The subject also fit-checks the collar using the size-check feature and/or the force-check feature. The sensor data and fit-check data then may be transmitted to a separate computing device (e.g., a smart phone, tablet, laptop, or other computer) for display and/or analysis. In another embodiment, an operator other than a subject (e.g., a team trainer) fit-checks a plurality of collars from a plurality of individuals (e.g., team members) to ensure proper collar sizing and resiliency, and retrieves and stores the sensor data and the fit-check data in the memory unit of the fit-check device. Optionally, these data are later transmitted to a separate computing device for display and/or analysis. In this way, the trainer can quickly ensure that the safety device is functional and can obtain nearly real-time updates from the sensors in a single, convenient device. The sensor data may be retrieved from the collar and stored in the fit-check device memory unit either automatically when the collar is connected or brought within communication range of the fit-check device. Alternatively, the sensor data is transferred from the collar to the fit-check device upon a manual action by the subject or operator. In another embodiment, the system is configured such that the collar sensor data is downloaded directly to the separate computing device, without the intermediate steps of the sensor data being captured, stored, and retransmitted by the fit-check device.

The fit-check device preferably is contained in a rigid housing. The housing may be constructed of any suitable material but a rigid plastic, polymer, or metal (e.g., aluminum, steel, etc.) housing is preferred. Preferably, the fit-check device is generally square or rectangular in shape but any other suitable shape may be used.

In some embodiments, the fit-check device 300 further comprises a internal battery and/or a connection for an external power source. The internal battery may be rechargeable or replaceable/disposable. The power source, whether a battery or externally supplied, is in electrical connection with one or more of the various electrical components described herein.

### Gap Assessment Using A Measuring Device

In one embodiment, the fit-check device 300 has a size-check feature (i.e., a measuring device) that indicates to the subject or the operator whether the gap 110 has the appropriate size to ensure that the collar is correctly sized for the neck size of the subject. The measuring device may be configured to measure the gap size when the device is donned in its intended position.

In one embodiment, the measuring device is a simple ruler. The ruler may be demarcated in any convenient units or it may be provided merely as unitless striations. In operation, the operator either measures the absolute or relative distance between the terminals 140 or compares the location of the terminals 140 against the striations to determine whether the gap has a proper size, is too small (indicating that the collar is too large for the subject), or too large (indicating that the collar is too small for the subject). In a related embodiment, the measuring device is presented as a series of segments or target areas. Optionally, the segments or target areas are colored, shaded, or otherwise demarcated to aid in the interpretation of fit. As illustrated in FIG. 2, the measuring device 150 may contain five separate segments (151-155, respectively). In one example, the first, third, and fifth segments (151, 153, and 155, respectively) have a first color (e.g., red) and the second and fourth segments (152 and 154, respectively) have a second color (e.g., green). The measuring device 150 is aligned with the collar terminals 140. In one embodiment, a properly sized gap, indicating a properly sized collar, is indicated when one terminal 140a aligns with the second segment 152 and the other terminal 140b aligns with the fourth segment 154 simultaneously. The gap is indicated as being too small if both terminals 140 align within the third segment 153 or one terminal 140 aligns with the third segment 153 and the other terminal 140 aligns with either the second segment 152 or the fourth segment 154. The gap is indicated as being too large when one terminal 140 aligns with the first segment 151 and the other terminal 140 aligns with the fifth segment 155 or when one terminal aligns with the second segment 152 or the fourth segment 154 and the other terminal aligns with the fifth segment 155 or the first segment 151, respectively. When the subject is the operator using the measuring device according to these embodiments, the subject may view the measuring device in a mirror to determine the collar's fit. Accordingly, colored segments rather than rulers may be preferred.

In another embodiment, the second segment 152 and fourth segment 154 are replaced with contact sensors. Two contact sensors are provided on a single side or edge of the fit-check device 300 with a pre-determined spacing therebetween that is equal in distance to the gap 110 of a properly fitting collar 100. The contact sensors are operably linked to a power source and a fit indicator that provides a positive indication when each of the collar terminals 140 is simultaneously contacting the contact sensors. The contact sensors may be simple buttons that, when simultaneously depressed, complete an electrical circuit internal to the fit-check device 300, wherein the electrical circuit includes the power source and the fit indicator. Alternatively, the contact sensors may be electrical connectors that interface with electrical connectors provided on the collar terminals 140. In this embodiment, the collar is modified to include an electrical connector at each terminal and an electrical connection internal to the collar connecting the electrical connectors such that an electrical circuit is completed when the connectors on the terminals 140 contact the contact sensors on the fit-check device 300.

In some embodiments, the segments 151-155 or contact sensors may be slidably engaged on the side of the device and operably linked to a size selector input device such that the location of one or more segments or sensors is translocated based on the size selector input. For example, size selector input device may be a sliding mechanism that is controlled by the operator to select the collar size being tested which in turn translocates one or more segments or sensors to provide an appropriate spacing for that collar size and associated gap 110.

The fit indicator may take any form suitable to alert the subject or the operator that the collar terminals 140 are simultaneously contacting the contact sensors. Suitable fit indicators include, for example, a light (e.g., a light emitting diode; “LED”), a speaker that emits an audible tone, or a vibration generating device.

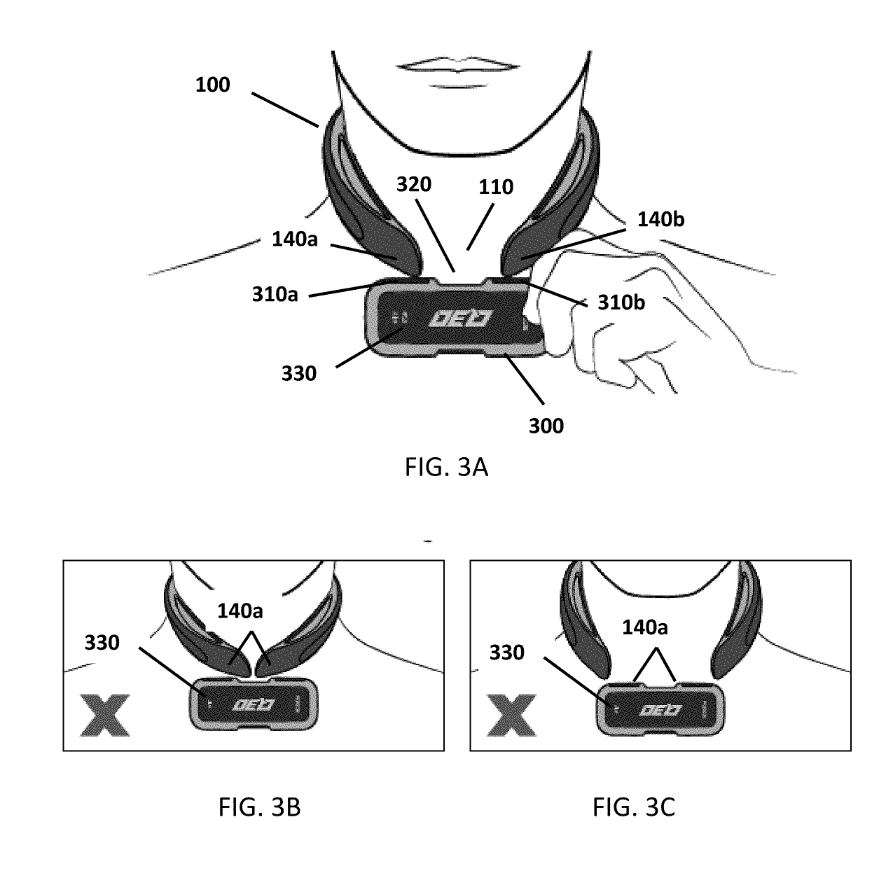

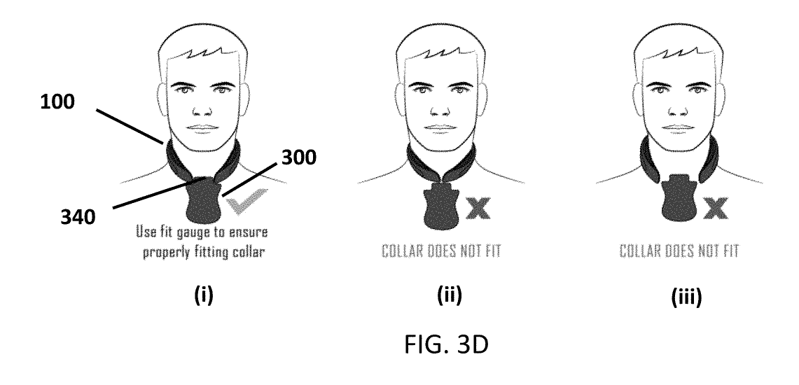

FIG. 3A illustrates an example in which a collar 100 is donned by the subject and the collar terminals 140a and 140b simultaneously contact a first contact sensor 310a and a second contact sensor 310b which are separated by a predetermined space 320. The simultaneous contact causes the fit indicator, illustrated as an LED 330, to illuminate. FIGS. 3B and FIG. 3C illustrate examples in which the collar is not appropriately sized to the subject. FIG. 3B illustrates the situation in which the collar is too large for the subject, rendering the terminals 140 too close together to simultaneously contact the contact sensors 310 such that the LED 330 remains unlit. FIG. 3C illustrates the situation in which the collar is too small for the subject.

In another embodiment, the size-check feature is provided on a face of the fit-check device. This configuration is particularly useful for fit-check devices that lack a straight edge of sufficient length to facilitate the gap measurement. For example, a round/disk-shaped or oval fit-check device may have the size-check feature on one face of the disk or oval.

In another embodiment, the fit-check device has an oval shape, wherein the long axis of the oval has a length equal to the length of the largest permissible gap 110 that indicates a properly fitting collar. The operator orients the oval-shaped device with the long axis extending across the gap 110 between the collar terminals 140 when the collar is donned. The collar is deemed too small for the subject if the collar terminals are not able to simultaneously contact the apex on each end of the oval. Alternatively, the collar may be deemed too small if the collar terminals 140 can contact each apex but with insufficient force to suspend the fit-check device against gravity. Optionally, the oval-shaped fit-check device has a width that is equal to the smallest permissible gap 110 that indicates a properly fitting collar. The collar is deemed as too large for the subject if the fit-check device is not able to pass between the collar terminals 140 without simultaneously contacting both terminals 140 when oriented with the oval width spanning the gap 110. In this way, the dimensions of the fit-check device provides the size-check feature without requiring any additional surface features (e.g., rulers and color bars) or sensors.

FIG. 3D illustrates a related embodiment in which the fit-check device 300 has a dimension 340 that falls within the range of acceptable gap 110 sizes. Preferably, the dimension 340 is equal to the smallest acceptable gap 110, the largest acceptable gap 110, or the midpoint between the largest and smallest acceptable gaps 110 when the collar 100 is donned. That dimension 340 of fit-check device is placed within the gap 110. The collar 100 size is deemed appropriate for the subject if the dimension 340 of the device 300 is capable of being placed within the gap 110 without requiring the subject to open or stretch the collar 100 and the collar terminals 140 simultaneously contact both ends of the dimension 340. Optionally, proper collar 100 sizing is indicated when the fit-check device 300 remains suspended after being released by the user (see, FIG. 3D(i)).

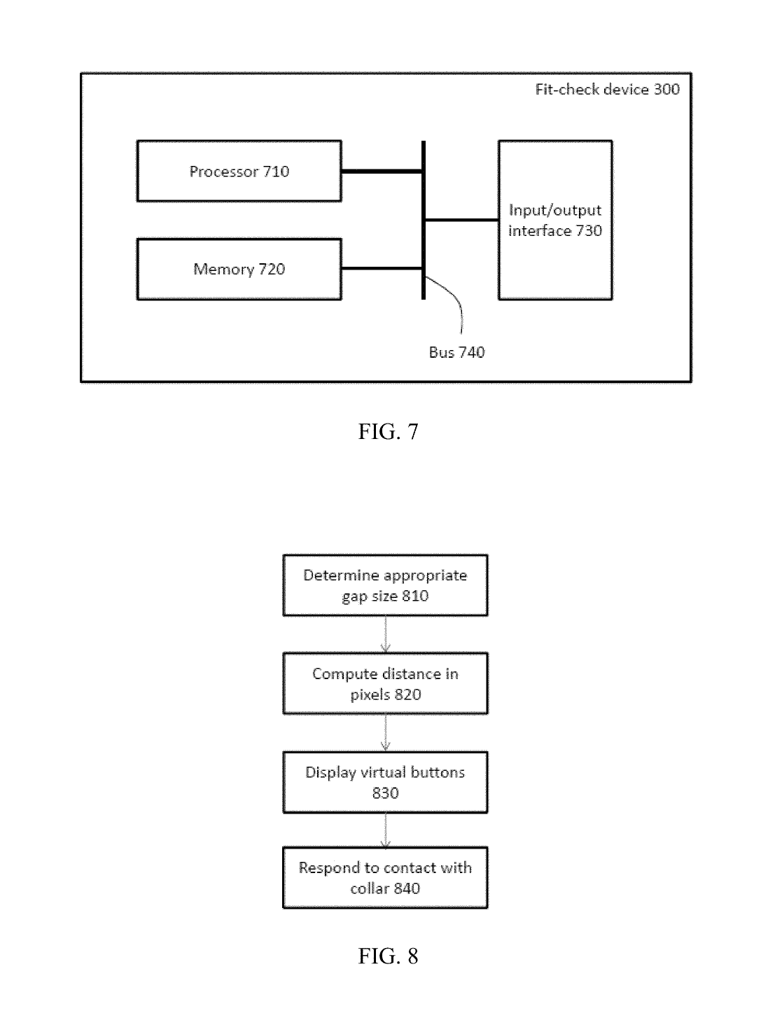

In an embodiment, shown with reference to FIG. 7, the fit-check device 300 contains a processor 710 (e.g., a microprocessor), a memory 720 and an input/output interface 730, all of which may be interconnected by a bus 740. The memory 720 stores computer-readable instructions that are executable by the processor 710. The input/output interface 730 may include one or more input devices and one or more output devices. The one or more input devices may include a screen (including a touch screen), a camera, an acoustic pressure transducer (e.g., microphone), one or more other (e.g., mechanical) pressure transducers, etc. The one or more output devices may include a screen, a loudspeaker, one or more lights (e.g., LEDs) and a mechanical transducer (to induce vibration), to name a few non-limiting possibilities.

The input/output interface 730 may also include a network interface for allowing the fit-check device 300 to communicate with an external device. In some embodiments, communication with the external device may be wired (tethered), in which case the network interface may be configured to implement a communication protocol (such as USB, for example). In other embodiments, communication with the external device may be wireless, in which case the fit-check device 300 may be equipped with a wireless transceiver and the network interface may be configured to implement a wireless protocol such as, for example, a near-field communication (NFC) protocol, Bluetooth or cellular (LTE, etc.) protocol. As such, it will be appreciated that depending on the embodiment, the fit-check device 300 may be stand-alone, tethered to a computer by a wire, or wirelessly networked. In some embodiments, the fit-check device 300 may be implemented using a smart phone, tablet or other networked wireless communications device.

Those skilled in the art will appreciate that the computer-readable instructions could be stored on a medium which is fixed and tangible (e.g., removable diskette, CD-ROM, ROM, fixed disk, USB drive) and readable directly by the processing entity, or the computer-readable instructions could be stored remotely but transmittable to the fit-check device 300 via a modem or other interface device (e.g., a communications adapter) connected to a network (including, without limitation, the Internet) over a transmission medium, which may be either a non-wireless medium (e.g., optical or analog communications lines) or a wireless medium (e.g., microwave, infrared or other transmission schemes) or a combination thereof. In other embodiments, elements of the fit-check device 300 may be implemented using pre-programmed hardware or firmware elements (e.g., application specific integrated circuits (ASICs), electrically erasable programmable read-only memories (EEPROMs), flash memory, etc.), or other related components.

Execution of the computer-readable instructions causes the fit-check device 300 to carry out various processes, including for processing the information received from the one or more input devices in accordance with an algorithm so as to cause the one or more output devices to impart effects or changes that are discernible by an operator of the fit-check device 300.

One such process, hereinafter referred to as a size-check process, may be described using the flowchart shown in FIG. 8. In one embodiment, the fit-check device 300 is implemented using a smartphone or tablet that is configured to implement a variety of software applications (“apps”) and the size-check process, which is carried out by the processor 710 of the fit-check device 300, may be triggered by activating one such app (a “fit-check app”) of the smart phone or tablet.

According to the size-check process, at step 810, the fit-check device 300 determines predetermined size criteria (e.g., an appropriate gap size or range of gap sizes) of a properly fitting collar. This can be achieved by retrieving the appropriate gap size from the memory 720. For example, this can be achieved by providing a data entry interface in which the operator can enter information about the collar such as the collar size or model number. The fit-check device 300 may use the operator-entered information to consult a lookup table having the appropriate gap size for difference collar sizes or model numbers. In a variant, the appropriate gap size, rather than being determined by the fit-check device 300, is manually entered by the operator into the fit-check device 300. Specifically, the operator may utilize a device other than the fit-check device 300 to access a server, provide the model number or collar size to the server, and obtain from the server a return message including the appropriate gap size (or range of gap sizes). Upon receipt of the appropriate gap size, the operator may enter this information into the fit-check device 300 via the data entry interface.

At step 820, the fit-check device 300 determines the screen size and resolution from hardware system data and converts the appropriate gap size into a pixel distance.

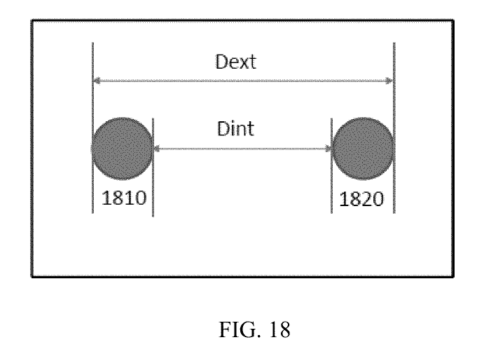

At step 830, the fit-check device 300 displays two virtual buttons on the touch screen, wherein the buttons may be linearly arranged and are separated by the pixel distance computed at step 820. In the case where step 810 involves the computation of a range of gap sizes, and with additional reference to FIG. 18, the buttons 1810, 1820 may each have a width such that the interior distance Dᵢₙₜ between the buttons 1810, 1820 corresponds to the gap size at the lower end of the range and the exterior distance Dₑₓₜ between the buttons 1810, 1820 corresponds to the gap size at the upper end of the range.

At step 840, the fit-check device 300 responds to contact between the collar and the touch screen. In an embodiment, the fit-check device 300 responds to contact between the collar terminals 140 and the touch screen. To this end, the collar terminals 140 and the touch screen are suitably configured to allow the touch screen to detect contact with the collar terminals 140. In an embodiment, the operator moves the touch screen to contact the collar terminals 140 when the collar is donned by the subject. The fit-check device 300 detects when the collar terminals 140 are simultaneously touching the buttons displayed on the touch screen and responds accordingly, by emitting a signal to the operator. The signal may be a visual signal, an audible tone, or a vibration, to name a few non-limiting possibilities. Optionally, the fit-check device 300 may emit a different signal if it detects that both collar terminals 140 are touching the screen but not simultaneously contacting the virtual buttons.

A variant of the above process is shown in FIG. 11 and may be applicable to embodiments of the fit-check device 300 implementing computer-readable instructions associated with a software application.

At step 1110, the fit-check device 300 determines the appropriate gap size (or range of gap sizes) of a properly fitting collar. This may be achieved in various ways.

In one non-limiting example, this can be achieved by providing a data entry interface in which the operator can enter information about the collar such as the collar size or model number. The fit-check device 300 may use the operator-entered information to consult a lookup table in the memory 720 which stores appropriate gap sizes for difference collar sizes or model numbers.

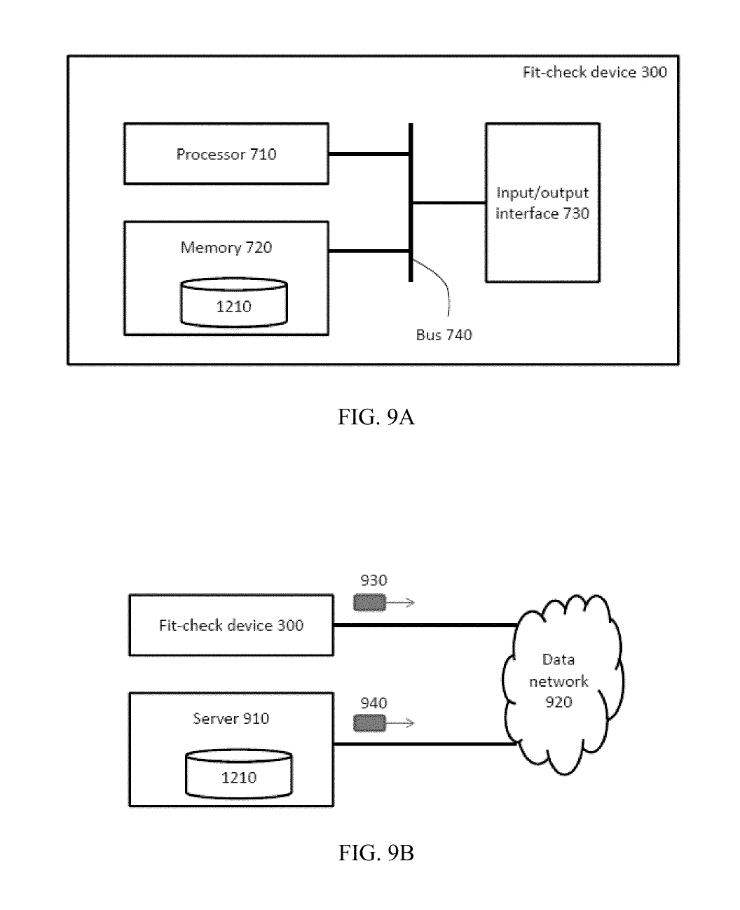

In another non-limiting example, the appropriate gap size is determined by providing a data entry interface (e.g., a graphical user interface) in which the operator can enter information about the subject, such as the subject's name, identification number, neck size, etc. The fit-check device 300 may use the operator-entered information to determine the appropriate pre-determined gap size. Specifically, and as shown in FIG. 12, the fit-check device 300 may consult a database 1210. The database includes a plurality of records 1220. Each of the records 1220 associates a subject (represented by a subject identifier) with a corresponding gap size. Examples of the subject identifier include the name of the subject, a subject ID number (e.g., team and number, or social security number) or even a biometric code. As specifically shown in FIG. 9A, the database 1210 may be stored in the memory 720 of the fit-check device 300. In other embodiments, and specifically as shown in FIG. 9B, the database 1210 may be stored at a server 910 reachable over a data network 920 such as the Internet. In such an embodiment, the fit-check device 300 establishes communication with the server 910 over the internet 920 and packages the subject identifier into a message 930 that is sent to the server 910 over the internet 920. At the server 910, the server 910 consults the database 1210 on the basis of the subject identifier (e.g., subject name or ID) to identify the appropriate one of the records 1220 and extract the associated gap size, which is then packaged into a return message 940 and transmitted back to the fit-check device 300 over the internet 920.

In further non-limiting example, the subject identifier, which is used to determine the appropriate gap size, is not entered by the operator of the fit-check device 300 or by the subject. Instead, the information about the subject may be autonomously collected by the fit-check device 300 itself. In one embodiment, this can be achieved by equipping the fit-check device 300 with an RFID reader, which reads an RFID tag worn by the subject.

In yet another non-limiting example, the appropriate gap size, rather than being determined by the fit-check device 300, is manually entered by the operator into the fit-check device 300. Specifically, the operator may utilize a device other than the fit-check device 300 to access the server, provide the subject identifier to the server and obtain the appropriate gap size from the server. Upon receipt of the appropriate gap size, the operator may enter this information into the fit-check device 300 via a user interface.

In yet a further non-limiting example, the appropriate gap size is determined based on measured physiological characteristics of the subject. For example, the fit-check device 300 is equipped with a camera and the camera is used to capture an image of the subject's neck from the front. The fit-check device 300 implements pattern recognition software in order to segment the image and locate the internal and external jugular veins of the neck. As a result, the distance between the two jugular veins may be obtained, and this distance can be related to the appropriate gap size by, e.g., multiplying by a certain coefficient.

At step 1120, the fit-check device 300 determines the actual size of the gap 110. This can be done in a variety of ways. In one embodiment, the collar terminals 140 are contacted with the touch screen. The collar terminals 140 and the touch screen are suitably configured to allow the touch screen to detect contact with the collar terminals 140. The operator may contact the touch screen to the collar terminals 140 when the collar is donned by the subject, but this is not a requirement. The fit-check device 300 then measures the distance, in pixels, between the contact points and may convert this into an absolute distance based on the screen size and resolution obtained from hardware system data.

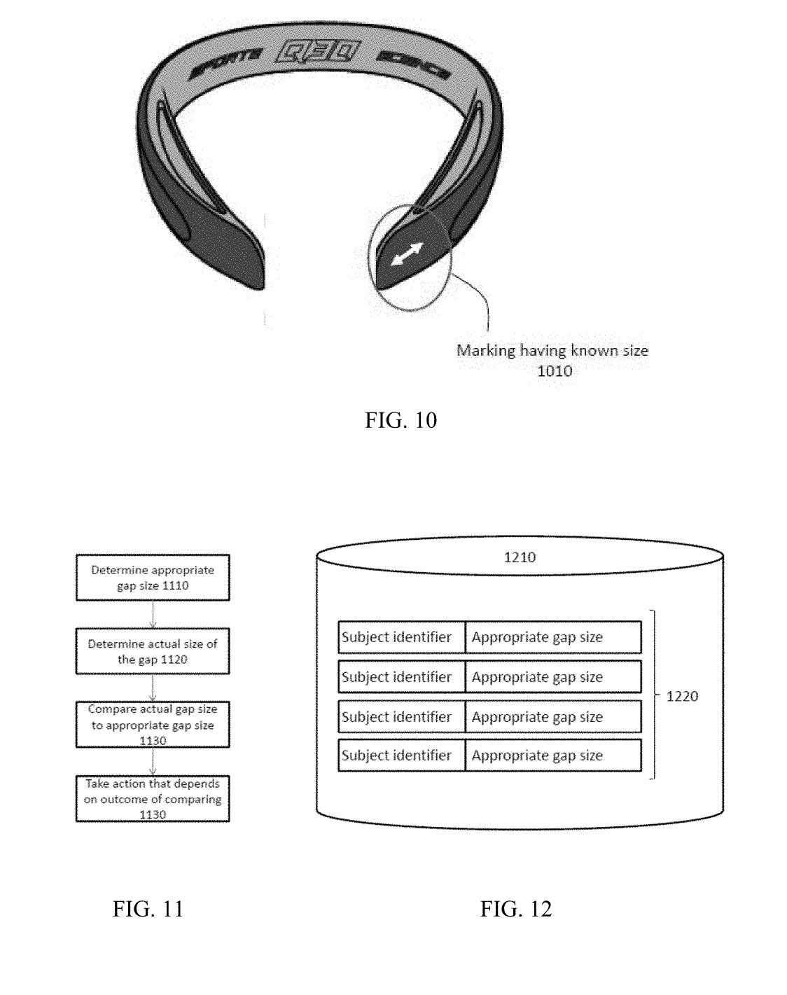

In a variant, the fit-check device 300 is equipped with a camera. The device is held in front of the subject with the camera aligned to the collar while the collar is donned. An image (picture) is taken of the front of the collar and the distance between the collar terminals 140 (i.e., the gap 110) is calculated from the resulting image. Optionally, as shown in FIG. 10, the collar also contains a visible marking 1010 of a pre-determined size (e.g., length) known to the fit-check device 300, and in a location that is expected to be present in the captured image. The software application may detect the visible marking 1010, determine its size within the image, and use the result to calibrate calculation of the actual size of the gap 110.

At step 1130, the actual gap size which was determined at step 1120 is compared to the appropriate gap size (or range of gap sizes) which was determined at step 1110.

At step 1140, the fit-check device 300 takes an action that depends on the outcome of the comparing which was performed at step 1130. For example, the fit-check device 300 may emit a first signal to the operator when the comparing yields that the actual gap size corresponds to the appropriate gap size (or is within the range of appropriate gap sizes), and a second signal when the comparing yields that the actual gap size does not correspond to the appropriate gap size (or is not within the range of appropriate gap sizes). The signals may be visual signals, audible tones, or vibration, to name a few non-limiting possibilities. One of these signals may also be the absence of a signal.

In a variant, the fit-check device 300 may issue a signal that provides the operator with an indication of the difference between the actual gap size and the appropriate gap size. Such indication may be in the form of volume, pitch, intensity of vibration, time spacing between consecutive beeps, etc.

In a further variant, when the actual gap size does not correspond to the appropriate gap size (or is not within the range of appropriate gap sizes), the fit-check device 300 may issue a signal that provides the operator with a suggestion. For example, where the actual gap size is smaller than the appropriate gap size, the fit-check device 300 may output the model number of a fit-check device that has a larger gap size. In another example, where the actual gap size is greater than the appropriate gap size, the fit-check device 300 may output the model number of a fit-check device that has a smaller gap size, or the fit-check device 300 may output a suggestion for one or more attachment elements that could be attached to the fit-check device 300 to adjust the size of the gap so that it does correspond to the appropriate gap size.

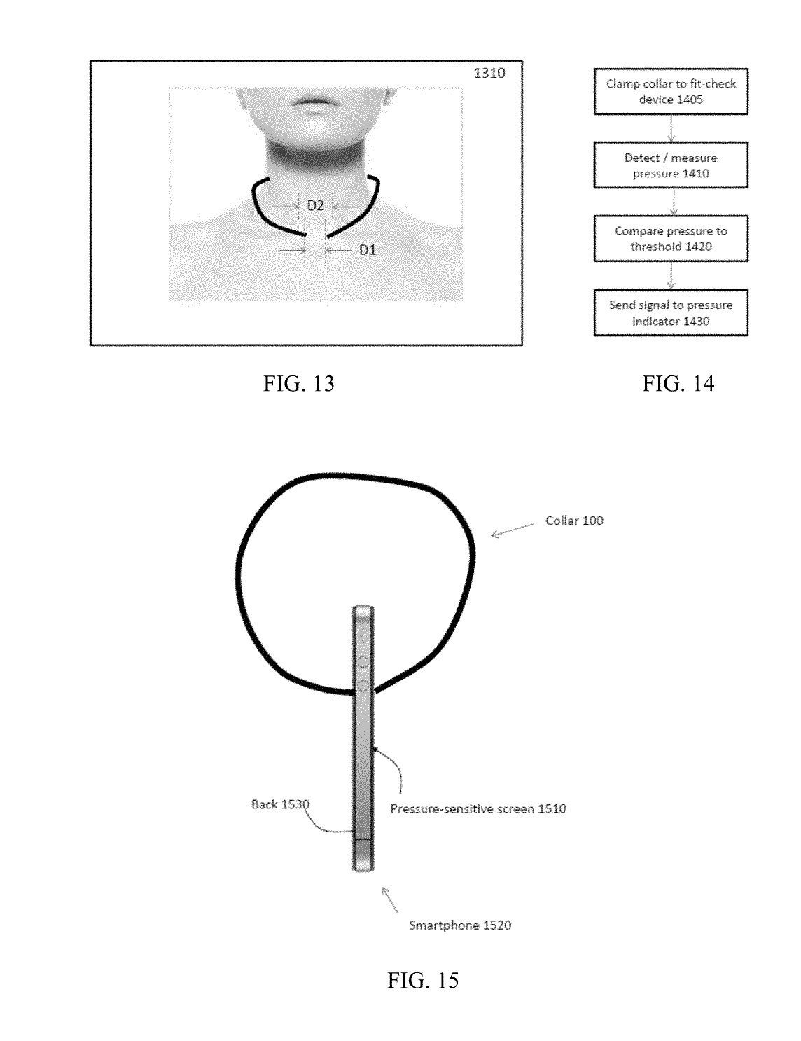

It should be appreciated that steps 1110 and 1120 may be performed in the described order, in the reverse order or in parallel. In a variant, both steps may be blended. For example, the fit-check device 300 is equipped with a camera. The device is held in front of the subject with the camera aligned to the collar while the collar is worn. An image (picture) is taken of the front of the collar from the front. With reference to FIG. 13, the image 1310 is processed by image processing software in order to detect the terminals 140 of the collar and the jugular veins of the subject. In this embodiment, the fit-check device 300 is computes the relative spacing between the jugular veins and the collar terminals 140. For example, the ratio of the distance D1 between the terminals 140 of the collar 100 and the distance D2 between the jugular veins may be denoted R (R=D1/D2).

Then, in lieu of the comparison at step 1130, the fit-check device 300 compares the ratio R to 1, which corresponds to perfect alignment between the collar terminals 140 and the jugular veins. Subsequently, actions similar to those described above in step 1140 may be taken as a result of this comparison.

It is understood that the length of the gap 110 varies based on the specific collar design and, therefore, the size and spacing of the various size-check features also will vary accordingly. However, it is preferred that the gap 110 of a properly fitting collar, when donned, is at least 1.5 inches. Smaller gaps may contact the subject's trachea, thereby reducing the comfort and possibly risking injury to the trachea. In one embodiment, the maximum acceptable gap for the indication of a proper fit is about 2.25 inches. Accordingly, the distance between the inner boundaries of the second and fourth segments of a color bar (i.e., the length of the third segment), contact buttons, or equivalent dimension of the fit-check device is about 1.5 inches. Likewise, the distance between the outer boundaries of the second and fourth segments of a color bar (i.e., the combined length of the second, third, and fourth segments), contact buttons, or equivalent dimension of the fit-check device is about 2.25 inches. Thus, each of the second and fourth segments or contact buttons has a length of about ⅜″.

### Collar Force Assessment Using A Pressure Sensor

In some embodiments, the fit-check device 300 has a force-check feature which is configured to measure the resiliency of the collar and/or its ability to apply an inwardly-directed force on the subject's neck veins. Generally, the force-check device consists of a first rigid side and a pressure sensor disposed on an opposing second rigid side. The pressure sensor detects and transmits the amount of force applied to a pressure transducer. The pressure transducer receives the input signal from the pressure sensor, interprets the amount of force applied to the pressure sensor relative to a predetermined desired amount of force (i.e., an amount that indicates a functional collar), and transmits an indication of pressure (e.g., display information) to the pressure indicator, which provides a signal to the operator. The length of the force-check device (i.e., the distance from the first rigid side to the pressure sensor) must be greater than the gap 110 of a functional collar 100. Typically, the force-check feature is used when the collar is doffed. Optionally, a second pressure sensor is disposed on the first rigid side, and a pressure input signal from both pressure sensors is integrated at the pressure transducer to indicate the total amount of inwardly-directed pressure exerted by the collar.

The pressure transducer may be a mechanical switch or spring that is activated upon the application of a predetermined amount of force. In one embodiment, the predetermined amount of force is equivalent to the amount of force necessary for the collar 100 to achieve sufficient occlusion of the neck vein(s) to effect TBI mitigation or prevention (i.e., sufficient to cause the application of about 5-80 mm Hg pressure on the neck vein(s)). In a related embodiment, the pressure transducer contains a second switch that is activated upon the application of a second predetermined amount of force that is greater than the first predetermined amount of force. The second predetermined amount of force is selected to represent an amount of force that would be unsafe if applied to the neck of a subject wearing the collar 100. A mechanical pressure transducer may include a simple electrical circuit that is completed/closed upon a sufficient application of force to the pressure sensor. The electrical circuit also includes the force indicator.

Alternatively, the pressure transducer may contain a microprocessor. In one embodiment, the microprocessor compares the applied amount of force applied to the pressure sensor to a look-up table, and engages a logic circuit to send the appropriate electrical signal to the pressure indicator. Here again, the microprocessor may be configured to generate one signal at one predetermined pressure or two different signals at two different predetermined pressures.

Optionally, the pressure transducer is in mechanical or electrical communication with a user input device. The user input device is configured to accept information about the collar (e.g., size, model number, etc.) and adjust the value for the predetermined amount of force required to trigger the various signals. The input device may be a toggle or sliding switch capable of selecting from among various collar sizes.

It is understood that the force measured by the force-check feature is not necessarily the same as, but is related to, the force that the collar applies to the neck veins. The disparity occurs because the force-check feature measures the inwardly-directed force exerted by the collar terminals 140, but the collar applies neck vein pressure from a different collar region (e.g., the protuberances). The inwardly-directed force applied by the collar terminals 140 may vary for any given neck-vein pressure based on the specifics of the collar construction. The relationship between the inwardly-directed collar terminal 140 force and the force applied to the neck veins may be easily determined. In one embodiment, a functional and properly-fitting collar having a gap 110 of about 1.5″-2.25″ exerts an inwardly-directed force of about 0.5-0.75 lbs. when measured between the collar terminals 140.

In other embodiments, the force-check feature may be designed to measure the inwardly-directed force generated between the portions of the collar that are designed to contact the neck veins (e.g., the protuberances). The fit-check device for this embodiment is constructed in accordance with the principles and features described herein.

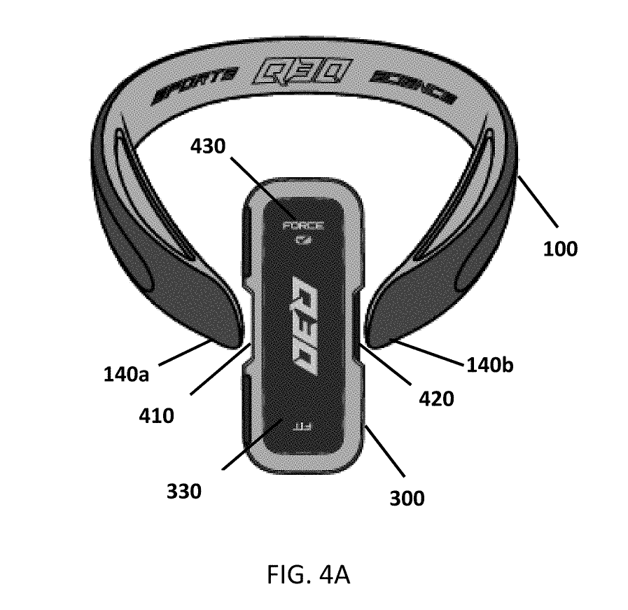

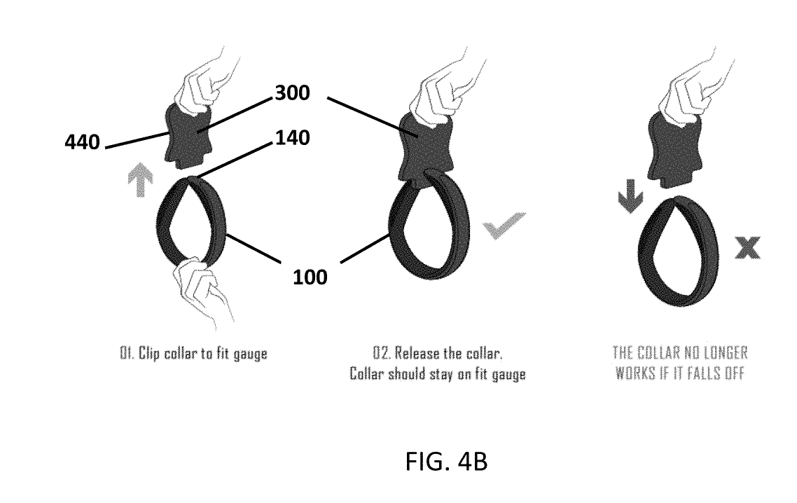

FIG. 4A illustrates the principles of the force-check feature. A fit-check device 300 is placed within the gap 110 of a doffed collar 100 such that a first collar terminal 140a contacts a first rigid side 410 of the fit-check device 300. The second collar terminal 140b contacts a pressure sensor 420 on a parallel/opposing side of the device 300. The fit-check device 300 detects the force applied to the pressure sensor 420 and transmits the appropriate signal to the pressure indicator. In this illustration, the pressure indicator 430 is illustrated as an LED which is illuminated only when sufficient force is applied to the pressure sensor 420.

Although the foregoing examples are described in terms of a fit-check device that is rectangular in shape or otherwise has at least one set of parallel sides on which the force-check feature is located, other device shapes are possible. For example, a round or disk-shaped fit-check device may have a pressure sensor and a collar-terminal contact point, or two pressure sensors, located at opposite sides of the circle, separated by the diameter. Oval devices may be similarly constructed with the collar terminal contact points spanning the width or length of the oval. Triangular devices are also possible wherein the contact points are located on each of two vertices such that the inwardly directed force is applied along one edge of the triangle.

FIG. 4B illustrates another embodiment of the force-check feature which is based on the physical dimensions of the collar and fit-check device, and does not require pressure sensors or other mechanical, electrical, or electronic components. The fit-check device has a dimension 440 that is larger than the gap 110 when the collar is doffed, which is smaller than the gap 110 when the collar 100 is donned. The fit-check device 300 is placed between the collar terminals 140 with the collar 100 being suspended below the device 300. The collar 100 is released while the operator holds the device 300. A collar 100 that applies an acceptable amount of inwardly-directed force will remain suspended from the device 300. A collar 100 that has lost some or all of its resiliency, such that it will not properly function when donned by the subject, will fall away from the device 300. In some embodiments, the surfaces or faces of the device 300 and/or the collar terminals 140 have a rubberized outer coating.

The pressure indicator is operably linked to the pressure transducer and is configured to output to the operator a signal indicating the amount of force applied to the pressure sensor either as an absolute value or relative to one or more pre-determined pressure values. The pressure indicator may be a digital screen or analog gauge. The displayed signal may be an absolute value of pressure, or it may be relative such as a gauge and needle, or other visible indictor such as “pass/fail,” “good/bad,” a check mark or an “X”. Alternatively, the pressure indicator may be a light (e.g., a light emitting diode; “LED”) or series of lights, a speaker that emits an audible tone, and/or a vibration generating device. The pressure indicator may utilize the same or a different display device as used for the size-check feature.

The force-check feature also may be implemented by virtue of the processor 710 executing the computer readable instructions stored in the memory 720. In this embodiment, the at least one output device of the fit-check device 300 includes one or more contact regions each equipped with a corresponding pressure sensing gauge connected to or comprising a microprocessor. The pressure sensing gauge may be integrated with a stand-alone fit-check device. Alternatively, the pressure sensing gauge may be implemented by a pressure-sensitive touch screen of a smartphone, such as is provided by Apple's iPhone6™ smartphone in a non-limiting embodiment. Still other examples of pressure sensors or pressure sensing gauges will occur to those of ordinary skill in the art.

An example force-check process is now described using the flowchart shown in FIG. 14. In one embodiment, the force-check process may be triggered by activating the same app on a smart phone or tablet as for the size-check process, or a different app may be activated.

With reference therefore to FIG. 14, at step 1405, the collar terminals 140 are somewhat separated from their at-rest position and positioned so as to clamp the fit-check device 300, thereby applying pressure to the one or more pressure sensors. Clamping may be done at contact regions specifically designed (e.g., indented or sensitized, etc.) to accommodate the collar terminals 140. In other embodiments, the contact regions may be represented by a flat surface with no specific physical or functional changes made to accommodate the collar terminals 140. The distance between the collar terminals 140 during application of pressure at step 1405 (i.e., the thickness of the fit-check device 300 between the contact regions) is assumed to be known to the fit-check device 300.

At step 1410, the fit-check device 300 measures/detects the pressure applied at the one or more pressure sensors of the fit-check device 300.

At step 1420, the fit-check device 300 compares the pressure detected by one or more pressure sensors to a threshold, or to a threshold range of pressures, or accesses a lookup table based on the sensed pressure.

At step 1430, the fit-check device 300 engages a logic circuit to send an electrical signal or message to the pressure indicator. The contents of the signal or message depend on the outcome of the comparison that was carried out at step 1420. For example, the microprocessor of the fit-check device 300 may be configured to send one signal at one pre-determined pressure or two different signals at two different pre-determined pressures. Alternatively, the microprocessor of the fit-check device 300 may be configured to send one signal when the detected pressure is within a first pre-determined range (e.g., corresponding to acceptable pressures) and a second signal when the detected pressure is outside the pre-determined range (e.g., which signals that the applied/detected pressure will be deemed unacceptable).

Those of skill in the art will appreciate that the acceptable pressures referred to above may be calibrated. That is to say, pressures detected at step 1410 may be considered “acceptable” even though they do not necessarily fall within the range of pressures that need to be applied by the protuberances to the neck veins of the subject so as to be effective. This is because the distance between the collar terminals 140 when taking the pressure measurement at step 1410 may be different (e.g., smaller) than the size of the gap 110 between the collar terminals 140 when properly worn by the subject. The detected pressure will be lower than the actual pressure that would be applied by the protuberances when the collar 100 is properly worn. Nevertheless, the detected pressure can be converted to an inferred neck pressure, and the inferred neck pressure compared to a desired neck pressure (or range of pressures). Alternatively, the detected pressure at the collar terminals 140 can be compared to a converted terminal pressure that has been derived from the desired neck pressure but calibrated to the (shorter) distance between the collar terminals 140 when the pressure measurement is taken.

Of course, in other embodiments, the need for determining an inferred neck pressure from a collar terminal pressure can be obviated by separating the collar 140 (so that the protuberances become separated by a distance corresponding to the distance that would separate them, if the collar 140 had been applied to the subject's neck) and then measuring the pressure between the protuberances. In this way, the pressure measurement directly corresponds to the pressure that would have been applied to the neck and a more straightforward comparison can be made with a predetermined pressure or range of pressures considered to be acceptable for the TBI-mitigating collar and/or the subject.

With reference to FIG. 15, it will be appreciated that the one or more pressure sensors may include a single pressure transducer such as is implemented, for example, by a pressure sensitive screen 1510 of a smartphone 1520. In this embodiment, the smartphone 1520 executes a software application (app) that is capable of measuring pressure applied to the screen 1510. In this case, step 1405 involves placing the collar terminals 140 so that one of them applies pressure to the touch screen 1510 and the other presses against a back 1530 of the smartphone. That is to say, the smartphone 1520 is clamped, along the thinnest of its dimensions, by the collar terminals 140. To this end, the smartphone 1520 may even be equipped with a case (not shown) that has a notch to facilitate engagement of one of the collar terminals 140 with the back 1530 of the smartphone 1520. It should be noted that the term smartphone is not used in a limiting sense, as it could refer to a tablet or other flat screen communication device.

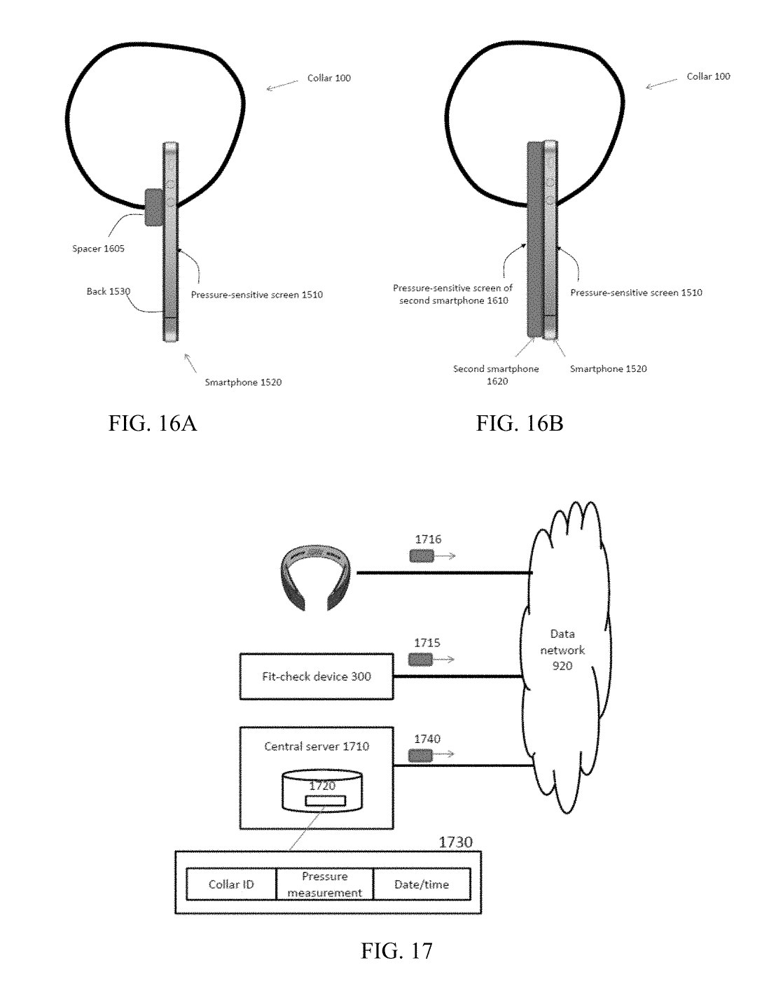

However, since the thickness of the smartphone 1520 may be considerably less than the distance between the collar terminals 140 when the collar 100 is properly worn, it is possible that the pressure reading made by the fit-check device 300 (smartphone 1520) under such circumstances may not be amenable to accurate calibration to an inferred neck pressure in the case where the collar 100 would be properly worn and the collar terminals 140 would be separated by a wider gap. This is because for thin smartphones (e.g., the smartphone 1520), the pressure applied to the touch screen 1510 by the collar 100 may simply be too insignificant. For this reason, and as shown in FIG. 16A, a spacer 1605 may be provided, so as to increase the gap between the collar terminals 140, which will cause the pressure applied by the collar terminals 140 to the touchscreen 1510 to be increased. If the thickness of the smartphone 1520 is known and the appropriate gap distance for proper use is also known, then it may even be possible to choose the spacer 1605 with a thickness that will make up for the difference in thickness, thereby obviating the need for calibration.

It should be appreciated that the smartphone 1520 may be configured to respond in various ways to determining that the applied pressure corresponds to a predetermined threshold or range, including by emitting the sound, flashing the screen or producing any other number of discernible effects or changes.

In a variant, as shown in FIG. 16B, the fit-check device 300 may be implemented as two flat screen communication devices (e.g., smartphones or tablets) 1520, 1620 placed back-to-back, leaving their respective pressure sensitive screens 1510, 1610 exposed. The collar 100 is then clamped to this “smart phone sandwich”. In particular, each of the collar terminals 140 is placed on a respective one of the screens 1520, 1620. In this way, not only does the second device 1620 act as a spacer, it also provides a pressure-sensitive screen, which effectively allows two pressure transducer readings to be obtained, thereby possibly leading to increased accuracy when calculating the applied pressure. An app running on one or the other of the smartphones 1520, 1620 may calculate the applied pressures and calibrate them, if necessary, before providing an indication of whether the collar 140 is exerting a suitable amount of pressure. In one configuration, one of the apps may be selected to be the “master” and the other may be the “slave”, which is controlled by the master. A spacer of any suitable size (of which the app would be informed) could be added between the sandwiched devices to further increase the applied pressure and/or better approximate the appropriate gap distance.

Here again, based on the detected pressure, the app may suggest modifications to the collar (e.g., expansion pieces or other adjustments) that would make the applied pressure more suitable.

In a further variant, the collar 100 itself is equipped with a measurement device (e.g., pressure sensor, (blood) flow sensor, etc.) at the collar terminals 140 or protuberances, as well as communications capability (e.g., NFC, Bluetooth or other wireless communication technology). In such an embodiment, pressure measurements are made by the collar 100 and communicated to the fit-check device 300, which performs the comparison at step 1420 and the outputting of a signal at step 1430. In this case, the pressure measurements obtained may be actual neck pressures, and thus calibration and conversion into inferred neck pressure may not be required.

In one embodiment, sensors regardless of how and where the pressure measurements are made, such measurements (e.g., pressure for a given gap distance) may be stored in memory of the fit-check device 300 and associated with the make, model and/or serial number of the collar from which they were generated. This information (i.e., the association between the make, model or serial number of the collar 100 and the measured pressure at a given gap distance) may be stored in the memory 720 of the fit-check device 300 and extracted from the memory 720 of the fit-check device 300 by an external computer, e.g., via a USB port. Alternatively, and with reference to FIG. 17, the information regarding the pressure measurements and the collar 100 may be communicated by the fit-check device 300 to a central server 1710, for example in the form of messages 1715 communicated over the aforementioned communication network 920 which may be the internet. Such information may be communicated to the central server 1710 directly by the collar 100, e.g., in the form of messages 1716, without interference or assistance from the fit-check device 300.

The central server 1710 may be associated with a manufacturer of the collar 100. The central server 1710, upon receipt of messages 1715 and/or 1716 communicating pressure measurements taken at different periods of time in the life of the collar 100, may store this information in a database 1720. The database includes records 1730, each record including a collar ID (e.g., make, model or serial number) of a particular collar, which is in association with a pressure measurement taken for a reference gap distance and also in association with a date/time when the measurement was carried out. In this way, it is possible for the central server 1710 to track pressure measurements taken over time and, by applying a data mining function to this historical data in the records 1730, the central server 1710 may be able to identify trends in the applied pressure over time. In particular, it may be possible for the central server 1710 to look out for and identify an inflection point in a downward trend in pressure of a particular collar, which may be interpreted to mean that the particular collar has started to lose its resiliency. This, in turn, may lead the central server 1710 to issue a message 1740 to a registered owner/operator of the collar 100 (e.g., in the form of an email message, SMS message, etc. sent to a device associated with the owner/operator, or directly to the collar itself), indicating that there is an increased risk that the collar 100 will not function properly. In some cases, this knowledge may be used by the manufacturer of the collar 100 to deliver enhanced customer service. In some cases, this may facilitate managing a recall of the collar 100.

### Fit-Check Device Construction and Design

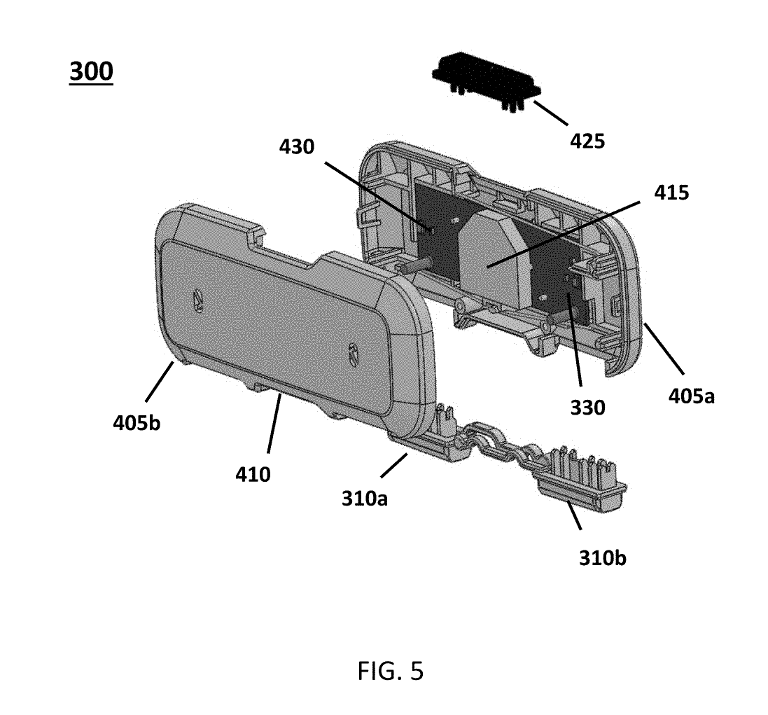

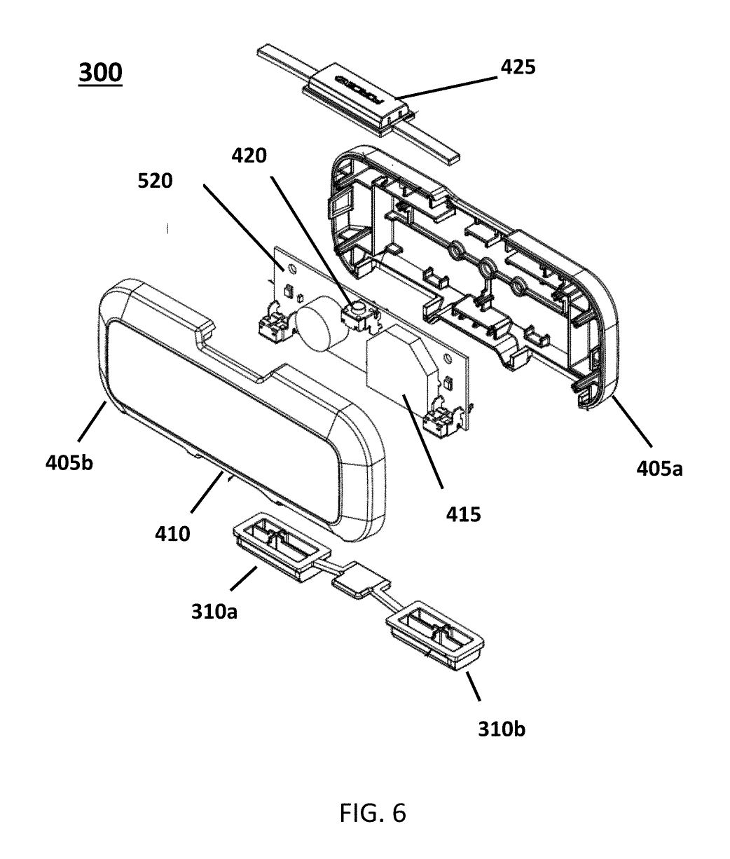

FIGS. 5 and 6 illustrate the construction principles of one embodiment of a fit-check device 300. The fit-check device 300 generally has an outer shell which may be fabricated as two or more separate pieces (405a and 405b) prior to final assembly. The force-check feature is illustrated as spanning the shortest dimension of the rectangular device, wherein the outer shell pieces 405a and 405b form the first rigid side 410. The pressure sensor 420 is housed below the pressure sensor cover 425 and is in functional communication with the pressure transducer 415, which itself is in electrical communication with the pressure indicator 430, illustrated as an LED. FIG. 6 illustrates an internal printed circuit board assembly 520 which may house and functionally connect the pressure sensor 420, the pressure transducer 415, a memory unit, a speaker, and/or a battery.

### Systems and Devices

The foregoing disclosure defines a fit-check device for use in conjunction with a partially circumferential concussion-mitigating collar. In some embodiments, the invention includes a fit-check device having the size-check feature, the force-check feature, or both features. In other embodiments, the fit-check device also has a memory unit and a data communication device to retrieve sensor data from the collar and transmit stored data (including sensor data from the collar and fit data from the fit-check device) to another device/processor for display and/or analysis. In other embodiments, the invention provides a software application for touchscreen devices that are capable of performing a size-check.

Thus, the invention also provides systems that include a partially-circumferential collar and a fit-check device and/or a size-check software application. Optionally, the system also may include software or other aids to analyze, interpret, and/or display sensor data and/or fit data.

As a person skilled in the art will recognize from the previous detailed description and from the drawing FIGS., and from the claims set forth below, modifications and changes may be made to the embodiments of the present application without departing from the scope of this present application as defined in the following claims. Although the foregoing examples have been described in some detail for purposes of clarity of understanding, the above-described inventive techniques are not limited to the details provided. There are many alternative ways of implementing the above-described techniques or the present application. The disclosed examples are illustrative and not restrictive.

1. A device comprising:
(a) a housing comprising a first rigid side and an opposing second rigid side,
(b) a pressure sensor positioned on the first rigid side and adapted to confirm an amount of compressive force between the first and second rigid sides,
(c) a pressure indicator, and
(d) a pressure transducer configured to receive a pressure input signal from the pressure sensor and transmit a pressure output signal to the pressure indicator.

2. The device of claim 1, wherein the device further comprises a battery operably linked to the pressure sensor and the pressure indicator.

3. The device of claim 1, wherein the pressure transducer comprises a microprocessor.

4. The device of claim 1, wherein the pressure indicator comprises one or more light emitting diodes.

5. The device of claim 4, wherein the pressure indicator comprises at least two light emitting diodes having different colors.

6. The device of claim 1, wherein the pressure indicator comprises an audio speaker or a vibration generating device.

7. The device of claim 1, wherein the device further comprises a measuring device on a third side.

8. The device of claim 7, wherein the measuring device is a ruler.

9. The device of claim 7, wherein the measuring device is a color bar comprising at least five color segments.

10. The device of claim 9, wherein a first segment, a third segment, and a fifth segment are colored in a first color, and a second segment and a fourth segment are colored in a second color.

11. The device of claim 7, wherein the measuring device further comprises:
(i) two sensors positioned on the housing and separated by a pre-determined space; and
(ii) a fit indicator configured to provide a user with an indication of whether the two sensors are simultaneously activated.

12. The device of claim 11, wherein the two sensors are positioned on the same side of the housing.

13. The device of claim 11, wherein the fit indictor comprises one or more light emitting diodes, an audio speaker, or a vibration generating device.

14. (canceled)

15. The device of claim 1, wherein the device further comprises a data communication device operably linked to a memory unit.

16. The device of claim 15, wherein the data communication device is selected from the group consisting of a data port and a wireless transceiver.

17. A device comprising:
(a) a housing;
(b) two sensors positioned on the housing and separated by a pre-determined space, and
(c) a fit indicator configured to provide a user with an indication of whether the two sensors are simultaneously activated.

18. The device of claim 17, wherein the two sensors are positioned on the same side of the housing.

19. The device of claim 17, wherein the device comprises an electrical circuit that is completed when the two sensors are simultaneously activated.

20. The device of claim 17, wherein the fit indictor comprises one or more light emitting diodes, an audio speaker, or a vibration generating device.

21. (canceled)

22. The device of claim 17, wherein the device further comprises a data communication device operably linked to a memory unit.

23-34. (canceled)
